(19) United States
(12) Patent Application Publication
(10) Pub. No.: US 2006/0069436 A1
(43) Pub. Date: Mar. 30, 2006

(54) Trial disk implant

(75) Inventors: Jeffrey K. Sutton, Medway, MA (US); Michael O'Neil, W. Barnstable, MA (US); Kristy Lynn Davis, Smithfield, RI (US); Hassan Serhan, South Easton, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: DePuy Spine, Inc., Raynham, MA (US)

(21) Appl. No.: 10/957,410

(22) Filed: Sep. 30, 2004

(51) Int. Cl. A61F 2/44; A61F 2/46; A61B 5/103

(52) U.S. Cl. 623/17.13; 623/17.15; 606/102; 600/587

(57) ABSTRACT

A trial intervertebral disk implant includes a first plate, a second plate, adjacent to the first plate, a conformable layer between the first and the second plates, and a pressure sensor within the conformable layer. The pressure sensor measures a distribution of compression force exerted by the first and the second plates on the conformable layer. The trial implant includes indicating means for indicating a position of the first and the second plates relative to each other, and locating means, for locating a position of the trial implant relative to the vertebrae between which said trial implant has been placed. The trial implant further includes at least one retractable member, connected to at least one of the first and the second plates. The retractable member can be extended or retracted through an aperture defined by a surface of the plate that is proximal to an abutting vertebra.

### BACKGROUND OF THE INVENTION

The human spinal column consists of discrete, sequentially coupled bones (vertebrae) cushioned by cartilaginous spacers, referred to as intervertebral disks, disposed between opposing vertebral endplates. Intervertebral disks are elastic, allowing the spine to retain a high degree of flexibility. Failure of an intervertebral disk usually requires surgical intervention that may include implantation of artificial disks or other devices that restore the height of the spinal column and a natural angle between the adjacent vertebrae. To prepare the intervertebral space for implantation of an artificial disk, the surgeon removes the damaged disk material, distracts the adjacent vertebrae and, once the proper gap between the adjacent vertebrae has been created, inserts an implant.

Distraction of an intervertebral space necessary to create clearance sufficient for insertion of the disk implant results in potential misalignment of the implant with the vertebral endplates. The surgical procedure can be further complicated by techniques that obstruct the surgeon's field of vision, thereby impeding, in particular, the determination of the correct disk implant size. A need exists for a device and method that would facilitate determination of the correct size of the intervertebral space and suitable alignment of the disk implant that overcomes or minimizes these problems.

### SUMMARY OF THE INVENTION

The present invention relates to a trial intervertebral disk implant for use by surgeons in design and preparation of a permanent intervertebral disk implant. The invention also relates to a method of selecting an artificial intervertebral disk to be inserted between two adjacent vertebral endplates and to a method of identifying a location between two adjacent vertebral endplates for placement of an artificial intervertebral disk.

In one embodiment, the present invention is a trial intervertebral disk implant, comprising a first plate, a second plate, a conformable layer between the first and the second plates, and a pressure sensor within the conformable layer.

In another embodiment, the present invention is a trial intervertebral disk implant, comprising a first plate, a second plate, and a pressure sensor disposed at a surface of at least one of the first and the second plates proximal to an abutting vertebra.

In another embodiment, the present invention is a trial intervertebral disk implant, comprising a first plate, a second plate, and indicating means, disposed within at least one of the first or the second plate, for indicating the position of the first and the second plates relative to each other.

In another embodiment, the present invention is a trial intervertebral disk implant, comprising a first plate, a second plate, and locating means, disposed within at least one the first or the second plate, for identifying the position of the trial implant relative to the vertebrae between which said trial implant has been placed.

In another embodiment, the present invention is a trial intervertebral disk implant, comprising a first plate, a second plate, at least one retractable member, connected to at least one of the first and the second plates, and operating means, disposed in at least one of the first or in the second plate, for extending and retracting the retractable member. The retractable member can be extended or retracted through an aperture defined by a surface of the plate that is proximal to an abutting vertebra.

In another embodiment, the present invention is a method of selecting an artificial intervertebral disk to be inserted between two adjacent vertebral endplates, comprising the steps of inserting between two adjacent vertebral endplates a trial intervertebral disk implant. The disk implant includes a first plate, a second plate, a conformable layer between the first and the second plates, and a pressure sensor within the conformable layer. Further steps include measuring a distribution of compression force exerted by the endplates between which the trial implant has been inserted and comparing the measured distribution of compression force to a distribution that minimizes variation of distribution of compression force while supporting abutting vertebrae in a substantially correct position relative to each other to thereby select an artificial disk.

In another embodiment, the present invention is a method of identifying a location between two adjacent vertebral endplates for placement of an artificial intervertebral disk. The steps comprise inserting between two adjacent vertebral endplates a trial intervertebral disk implant. The trial implant includes a first plate, a second plate, at least one retractable member, connected to at least one of the first and the second plates, wherein the retractable member can be extended or retracted through an aperture in a surface of at least one of the first and the second plates proximal to an abutting vertebra, and operating means, disposed in at least one of the first and the second plates, for extending and retracting the retractable member. The steps further include extending the retractable member, thereby indenting at least one of the vertebral endplates between which the trial implant has been placed to identify the location for placement of an artificial intervertebral disk.

The present invention offers a number of advantages. A flexible layer allows extra degrees of freedom when inserted between the adjacent vertebral endplates. Pressure sensors can measure pressure distribution exerted by the endplates. The combination of these features allows the surgeon to design an optimally shaped permanent disk implant. Furthermore, the positional detectors that transmit the information about the location of the trial implant to the surgeon, reduces reliance of the personnel on fluoroscopy or X-rays thus minimizing exposure to harmful radiation.

### BRIEF DESCRIPTION OF THE DRAWINGS

FIG. 1A is an isometric view of a trial intervertebral disk implant of the instant invention.

FIG. 1B is a side view (partial cut-away) of the device of FIG. 1A.

FIG. 1C is a side view (partial cut-away) of the device of FIG. 1A in an angled conformation.

FIG. 2 is a schematic diagram of the device of FIG. 1A showing positioning of indicating and locating means.

FIG. 3 is a side view (partial cut-away) of an embodiment of the device of FIG. 1A where the angle between the plates is controllably adjustable.

FIG. 4A is an isometric view of one embodiment of a trial intervertebral disk implant of the present invention that includes retractable members.

FIG. 4B is a cross section view of the device of FIG. 4A.

FIG. 5 shows a rack-and-pinion mechanism that can be employed to extend and retract the retractable members.

FIG. 6 shows a worm gear mechanism that can be employed to extend and retract the retractable members.

FIG. 7 shows a detail of the retractable member and one of the plates of one of the embodiments of the trial implant of the present invention.

FIG. 8 shows a variable thickness cam mechanism that can be employed to extend and retract the retractable members.

FIG. 9A is an isometric view of one embodiment of the trial implant of the present invention that allows the retractable members to rotate.

FIG. 9B shows detail of the rotatable retractable members of the embodiment of FIG. 9A.

FIG. 10A shows a side view of one of the embodiments of the trial implant of the present invention that employs detachable actuator arms to deploy angle adjustment mechanism, retractable member operating mechanism and retractable member rotating mechanism.

FIG. 10B shows the device of FIG. 10A with an adjusted angle and an extracted retractable member.

FIGS. 11A through 11D show an embodiment of the conformable layer of the present invention having impedance sensors embedded therein and operating as a proximity sensor.

FIGS. 12A and 12B show alternative embodiments of a trial implant of the present invention.

FIGS. 13A and B show an alternative embodiment of a variable thickness cam mechanism of FIG. 8 that can be employed to extend and retract as well as to ritate the retractable members.

### DETAILED DESCRIPTION OF THE INVENTION

The foregoing and other objects, features and advantages of the invention will be apparent from the following more particular description of preferred embodiments of the invention, as illustrated in the accompanying drawings in which like reference characters refer to the same parts throughout the different views. Elements having the same number in different figures represent the same item. The drawings are not necessarily to scale, emphasis instead being placed upon illustrating the principles of the invention.

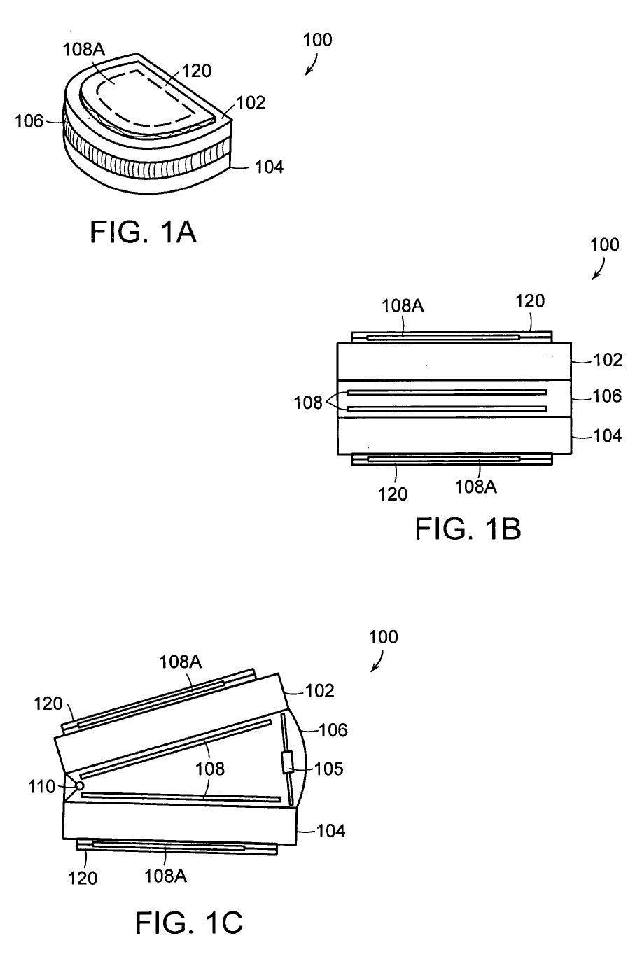

Referring to FIGS. 1A through 1C, in one embodiment, the present invention is a trial intervertebral disk implant 100 comprising first plate 102, second plate 104 adjacent to first plate 102, conformable layer 106 between first and second plates 102 and 104, and one or more sensors 108 within conformable layer. Sensors 108 can be selected from pressure, angle or distance sensors, e.g. proximity.

Examples of suitable materials of construction of plates 102 and 104 are stainless steel, titanium, cobalt chromium alloys, polymers such as polysulfone, polyethyeretherketone (PEEK), polyacetals, etc, or ceramics. As used herein, the term “conformable layer” means a layer that is elastic and returns to its original shape once the pressure is removed. Conformable layer 106 can be made from any suitable biologically inert elastic material, such as silicone, latex, rubber or urethane. Conformable layer 106 preferably allows first and second plates 102 and 104 to move substantially in any direction with respect to each other within the limits imposed by the elastic material of conformable layer 106.

Additionally, some embodiments of the trial implant of the present invention, not shown, do not include conformable layer 106. In these embodiments, plates 102 and 104 are held together by hinge 110 or any of the angle or height adjusting mechanisms described below.

FIG. 1C depicts plates 102 and 104 being at an angle with respect to each other by employing angle adjusting means 105. In some embodiments, hinge 110 is provided that allows plates 102 and 104 to assume an arbitrary angle relative to each other and arbitrary distance from each other, but not to slide parallel to each other. In other embodiments plates 102 and 104 can also slide parallel to each other. In one embodiment, pressure sensors 108 measure a distribution of compressive force exerted by first and second plates 102 and 104 on conformable layer 106.

Trial implant 100 includes layer 120 disposed at a surface of at least one of plates 102 and 104 proximal to an abutting vertebra. Sensors 108A, selected from pressure, angle or distance sensors (e.g. proximity sensors), can be embedded in layer 120, similarly to placing sensors 108 in conformable layer 106. In the embodiment where sensors 108A are pressure sensors that can measure a distribution of a compression force exerted on plates 102 and 104 by the abutting vertebrae. In some embodiments, layer 120 is a deformable layer, wherein deformation retained following extraction of the trial implant indicates the distribution of pressure on the trial implant when between vertebrae. In another embodiment, layer 120 is a conformable layer. As used herein, the term “deformable layer” means a layer that is not elastic and retains its shape after application of pressure. Examples of material suitable for use in layer 120 include silicone, latex, rubber, urethane or solid polymeric open cell foam (i.e. a sponge).

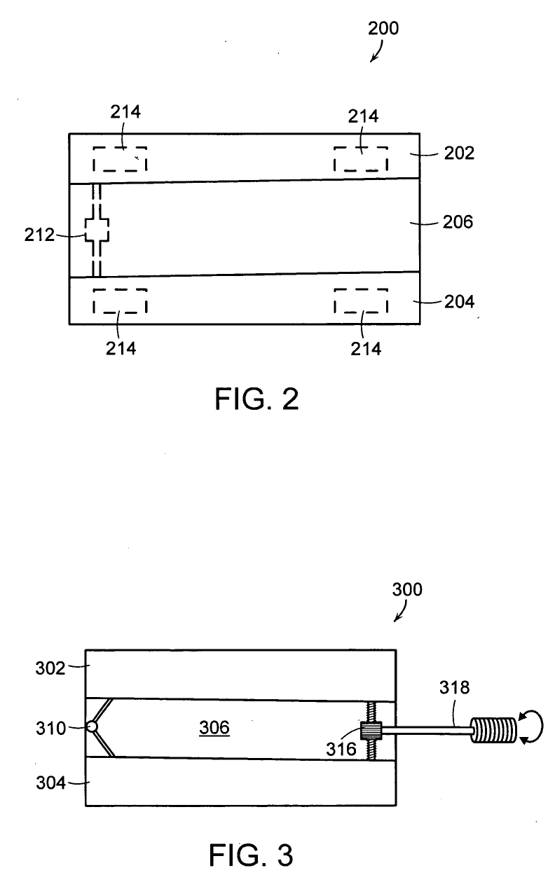

In another embodiment, represented by FIG. 2, trial implant 200 can further include indicating means 212 that can indicate a position of plates 202 and 204 relative to each other. Indicating means 212 can be disposed in either of the two plates 202 and 204 or within conformable layer 206, as shown in FIG. 2. Examples of mechanisms that can be used for the purpose of obtaining information regarding the angle between the plates, the proximity of the plates or the parallel translation of the plates with respect to each other are proximity sensors and Hall effect sensors as described below. One skilled in the art will be able to determine any other mechanism or device suitable for this purpose.

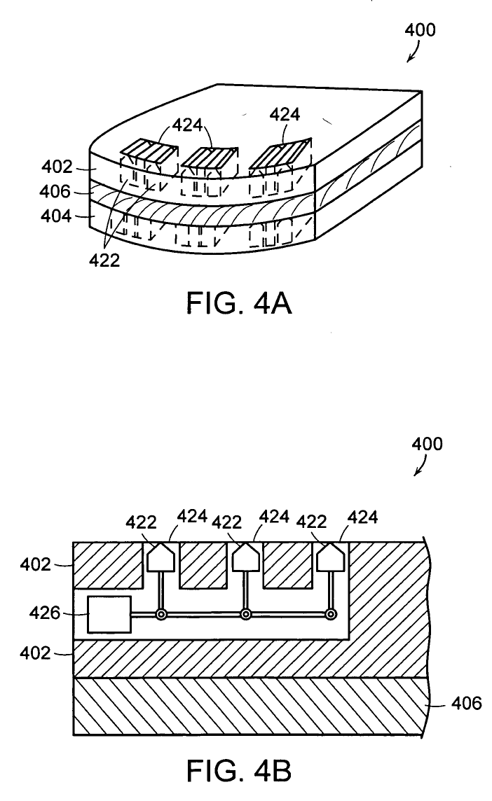

Trial implant 200 can include locating means 214 that can locate a position of device 200 relative to the vertebrae between which and surrounding tissue into which device 200 has been inserted. Locating means 214 can be disposed in either or both of the two plates 202 and 204, as shown, or within conformable layer 206. Locating means 214 can include an ultrasonic transducer, a tissue impedance sensor or an infrared (IR) proximity sensor. Locating means 214 transmit information about the position of device 200 to an processing device (not shown) that is used by the operating surgeon to locate device 200 within patient body without the use of harmful X-rays or fluoroscopy. For example, ultrasonic transducer can be used to generate ultrasound images of the tissues surrounding inserted device 200, impedance sensors can provide electrical feedback, such as impedance, and light-emitting diodes, preferably infra-red, and a photodetector to provide direct optical signal to help identify the location of device 200 and type of the native tissue, such as trabecular bone. cortical bone, nerve, collagen or cartilage. Determination of tissue density can be employed to determine the tissue type. Another embodiment of the trial implant is device 300 shown in FIG. 3. In this embodiment, the angle between first and second plates 302 and 304 can be controllably adjusted by employing angle adjusting means 316. Preferably, hinge 310 is provided that allows plates 302 and 304 to assume an arbitrary angle relative to each other, but not to slide parallel to each other. Conformable layer 306, similar to conformable layer 106 of device 100 can be provided. In the embodiment shown, angle adjusting means 316 is a jack screw device operated by the surgeon by rotating actuator arm 318, which can be detachable. In the alternative, other mechanical or electrical motive devices can be employed for this purpose.

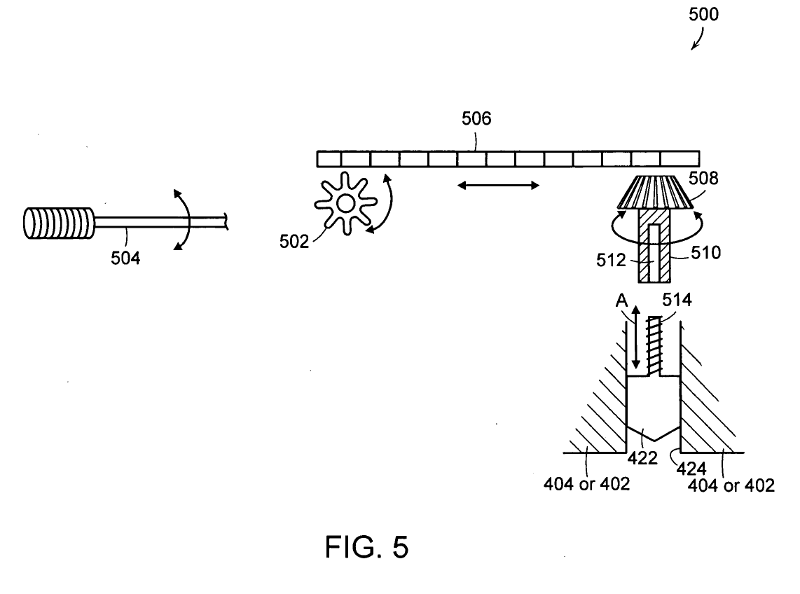

Referring to FIGS. 4A and 4B, in some embodiments, trial implant 400 includes at least one retractable member 422. Retractable members 422 are disposed in and may be connected to plates 402 and 404. Plates 402 and 404 can be separated by flexible layer 406, similar to layer 106 of device 100. Retractable members 422 are extended through apertures 424, which are defined by a surface of the plate that is proximal to an abutting vertebra. Retractable members 422 can indent vertebral endplates, between which trial implant 400 has been inserted, thus marking the position occupied by trial implant 400. Accordingly, in one embodiment, retractable members 422 can be employed as locating means 214 (see FIG. 2).

Referring to FIG. 4B, operating means 426 are used to extend or retract retractable members 422.

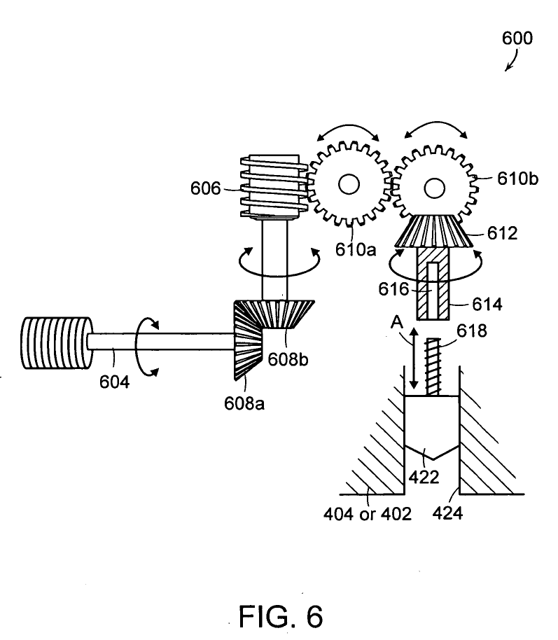

In one embodiment, shown in FIG. 5, operating means 426 is a rack-and-pinion mechanism 500. Mechanism 500 includes crown pinion 502, which can be rotated using any mechanical or electrical motive device, such as actuator arm 504. Actuator arm 504, in one embodiment, is detachable. Crown pinion 502 engages and moves rack 506, which, in turn, engages and rotates crown pinion 508. Crown pinion 508 includes threaded shaft 510, which defines an internally threaded bore 512. The assembly of pinion 508 and shaft 510 can freely rotate but is linearly constrained. In this embodiment, retractable member 422 includes externally threaded shaft 514, which engages the internal thread of bore 512. Retractable member 422 is rotationally constrained by can move linearly in a direction parallel to arrow A. As a result of rotation of pinion 502, retractable member 422 can thus be retracted or extended through aperture 424 in either first or second plate 402 or 404.

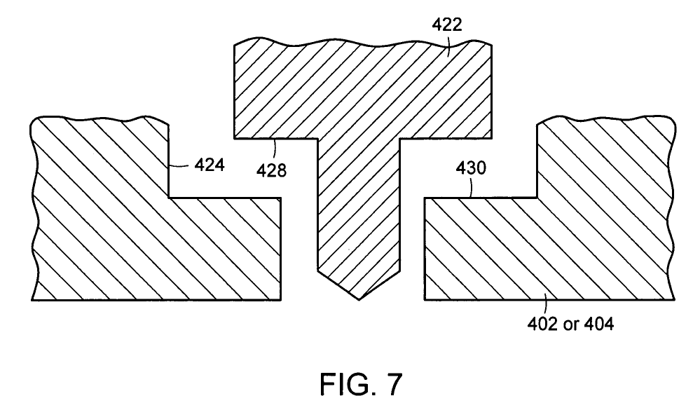

In another embodiment, shown in FIG. 6, operating means 426 is worm gear mechanism 600. Mechanism 600 can be engaged using any mechanical or electrical motive device, such as actuator arm 604. Actuator arm 604, in one embodiment, is detachable. Rotation of actuator arm 604 is transmitted to worm gear 606 through any known mechanism, for example a pair of conical crown gears 608a and 608b. Worm gear 606 engages crown pinions 610a and 610b, which in turn, transmit the motion to crown pinion 612, similar to previously described crown pinion 508 (see FIG. 5). Crown pinion 612 includes threaded shaft 614, which defines an internally threaded bore 616. The assembly of pinion 612 and shaft 614 can freely rotate but is linearly constrained. In this embodiment, retractable member 422 includes externally threaded shaft 618, which engages the internally threaded bore 616. Retractable member 422 is rotationally constrained by can move linearly in a direction parallel to arrow A. As a result of rotation of worm gear 606, retractable member 422 can thus be retracted or extended through aperture 424 in either first or second plate 402 or 404.

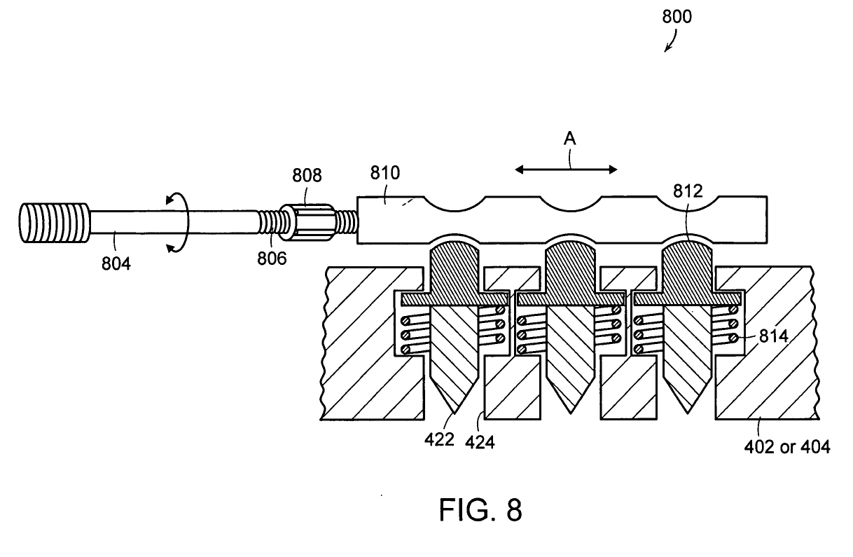

Referring to FIG. 7, when either rack-and-pinion mechanism 500 or worm gear mechanism 600 is used to operate retractable member 422, aperture 424 and retractable member 422 should preferably be shaped so as to include shoulder 428 on retractable member 422 and step 430 in aperture 424. This will prevent retractable member 422 from becoming fully disengaged during its extension.

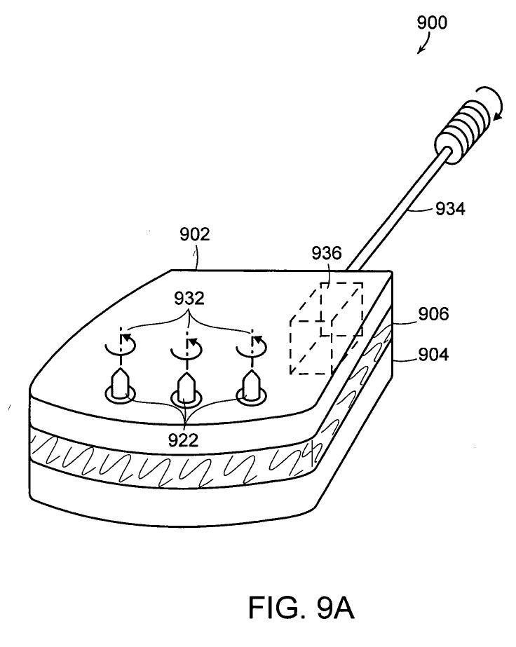

Another embodiment of operating means 426 is variable thickness cam mechanism 800, shown in FIG. 8. Mechanism 800 can be engaged using any mechanical or electrical motive device, such as actuator arm 804. In one embodiment, actuator arm 804 is detachable. In the example depicted in FIG. 8, actuator arm 804 includes a threaded portion 806 that is held by an internally threaded cuff 808, connected to one of the plates 402 or 404. Actuator arm 804 pushes onto variable thickness cam 810, which can move in a direction parallel to arrow A. As a result, the thicker portions of the body of cam 810 exert pressure on retractable members 422, pushing them through apertures 424 in plates 402 or 404. In this embodiment, retractable member 422 includes upper portion 812. Upper portion 812 has a shape that can slide on the surface of cam 810 while being pushed by the thicker portions of the body of cam 810. In the embodiment shown, upper portion 812 is spherically shaped. The assembly that includes retractable member 422 and upper portion 812 is spring-loaded by spring 814. Spring 814 facilitates retraction of retractable member 422.

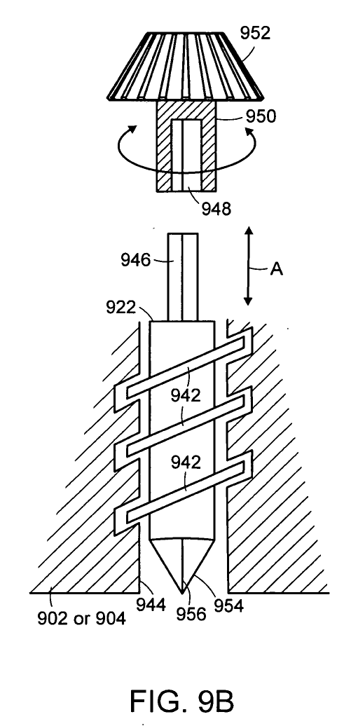

Another embodiment of the trial implant of the instant invention, device 900, is shown in FIG. 9A. Similarly to the previously described embodiments (devices 100, 200, 300 and 400), device 900 comprises first plate 902, second plate 904 and conformable layer 906, adjacent to first and second plates 902 and 904. Device 900 further includes one or more retractable members 922, similar to retractable member 422 (see FIGS. 4A and 4B).

In this embodiment, retractable member 922 can rotate around a major axis 932, thereby operating as a drill bit. Actuator arm 934, which can be detachable, or any other mechanical or electrical motive device can be employed to engage rotating means 936 to rotate retractable members 922. The mechanisms that can be used as rotating means 936 will be described below. In one embodiment of device 900, operating means, similar to operating means 426 (not shown) are used to extend or retract retractable members 922. In another embodiment of device 900, retractable members 922 can be moved between retracted and extended positions by rotation around axis 932.

FIG. 9B shows an embodiment of a mechanism that can be employed to rotate retractable member 922. In this embodiment, retractable member 922 includes thread 942 that engages aperture 944 and ridged shaft 946. Ridged shaft 946 engages polygonal bore 948 in shaft 950 of crown pinion 952. The ridges of ridged shaft 946 prevent it from rotating while engaged with polygonal bore 948, while allowing motion in a direction parallel to arrow A. Similarly to other mechanisms described above, the rotating motion of actuator 934 is transmitted to crown pinion 952. Rotation of crown pinion 952 is transmitted to retractable member 922 due to engagement of ridges of ridges shaft 946 and polygonal bore 948. Rotation of retractable member 922 extends it through aperture 944, while simultaneously rotating retractable member 922, due to engagement of thread 942 with the thread within aperture 944.

In one embodiment, retractable member 922 includes tip portion 956 which is provided with ridges 956 to facilitate drilling through the abutting vertebral bone.

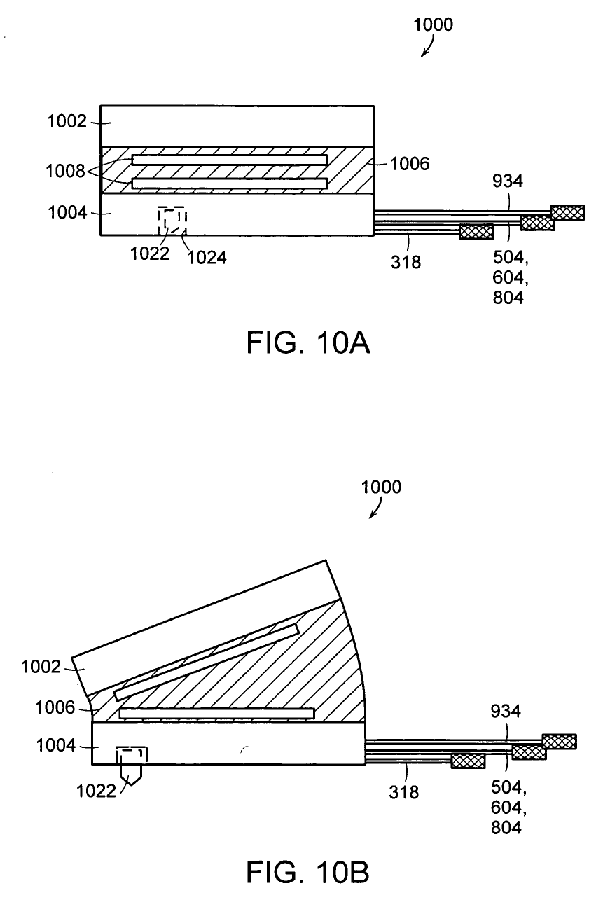

Any combination of the elements and mechanisms described above can be employed together in a trial intervertebral disk implant of the present invention. One embodiment of a trial implant of the instant invention, device 1000 is shown in FIGS. 10A and 10B.

Similarly to the previously described embodiments (devices 100, 200, 300, 400 and 900), device 1000 comprises first plate 1002, second plate 1004 and conformable layer 1006, adjacent to first and second plates 1002 and 1004. Pressure sensors 1008 are disposed in conformable layer 1006. Device 1000 further includes one or more retractable members 1022, extendable through aperture 1024, similar to retractable member 422 (see FIGS. 4A and 4B).

FIG. 10A shows trial implant 1000 with plates 1002 and 1004 parallel and retractable member 1022 retracted. FIG. 10B shows trial implant 1000 having the angle between plates 1002 and 1004 adjusted and retractable member 1022 extended. This embodiment includes angle adjusting means (not shown) similar to means 316 shown in FIG. 3, retractable member operating means (not shown) similar to means 426 shown in FIG. 4B, and rotating means (not shown), similar to means 936 shown in FIG. 9. Device 1000 is shown with actuator arms 318, 504, 604 or 804, and 934, attached.

Angle adjusting means can include means for changing the distance between plates 1002 and 1004. Such means can be selected from any of the mechanisms described above, described below or any other commonly known mechanisms, e.g. a wedge, an inclined plane, a screw, pressure chambers, magnetic field, etc.

Trial implant of the instant invention is particularly advantageous for selecting an artificial intervertebral disk to be inserted between two adjacent vertebral endplates. The operating surgeon, inserting between two adjacent vertebral endplates a trial implant of the present invention, can measure a distribution of compression force exerted by the endplates between which the trial implant has been inserted and can compare the measured distribution of compression force to a distribution that minimizes variation of distribution of compression force while supporting abutting vertebrae in a substantially correct position relative to each other. As used herein, the “substantially correct” position of the two vertebrae relative to each other refers either to a position of substantially natural lordosis or natural kyphosis, anterior-posterior position, medial-lateral position, and disk height. The angle between the two adjacent vertebrae can be positive, negative or zero (i.e., when the opposing surfaces of the adjacent vertebrae are essentially coplanar). By identifying the position of the trial implant relative to the vertebrae between which said trial implant has been placed, identifying the position of first and second plates 102 and 104 relative to each other and adjusting the angle between the upper and the lower vertebral endplates, the operating surgeon can determine the combination of the minimal variation of compression force and a substantially correct relative position of abutting vertebrae, thereby selecting a suitable permanent artificial disk implant.

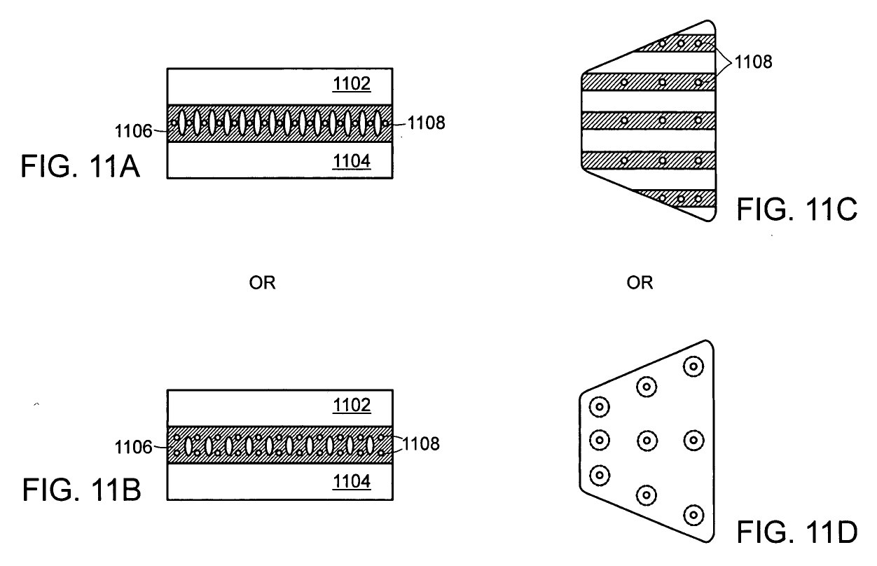

FIGS. 11A through D show alternative embodiments of a trial intervertebral disk implant of the present invention wherein conformable layer 1106, disposed between plates 1102 and 1104, includes impedance sensors 1108 that act as a proximity sensors. Specifically, as shown in FIGS. 11C and D, when plates 1102 and 1104 separate, the distance between individual impedance sensors 1108 and/or plates 1102 and 1104 increases, thus changing the current detected by each sensor. One skilled in the art will easily determine suitable impedance sensors and will be able to construct conformable layer 1106 as shown in FIGS. 11A-D.

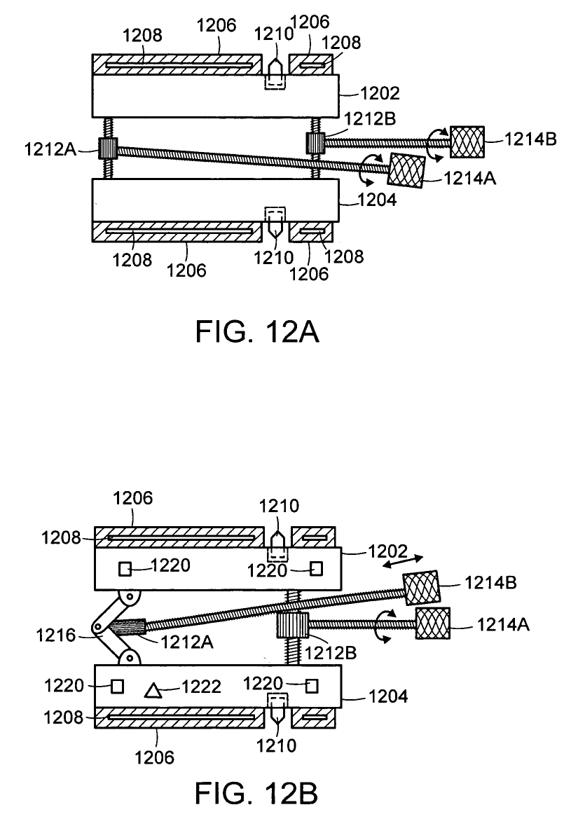

FIGS. 12A and B show an embodiment of a trial intervertebral disk implant of the present invention wherein conformable layers 1206 are disposed on the surfaces of plates 1202 and 1204 that are proximal to the abutting vertebrae. Sensors 1208, that can be a pressure sensor, an angle sensor, a distance sensor or a combination thereof are embedded in conformable layers 1206. Embodiments shown in FIGS. 12A and B further include at least one retractable member 1210, substantially similar to the retractable members described above. Angle adjusting mechanisms 1212A and 1212B, operable by actuators 1214A and 1214B are provided to adjust the distance and/or the angle between plates 1202 and 1204. Actuators 1214A and B can be detachable handles. In one embodiment, shown in FIG. 12B, hinge 1216 is employed to hold the plates together and to operate as a part of angle adjusting mechanism 1212A.

The embodiment of a trial intervertebral disk implant can further include Hall effect sensors 1220 (and optionally a magnet, not shown) and/or ultrasound sensor and/or emitter 1222.

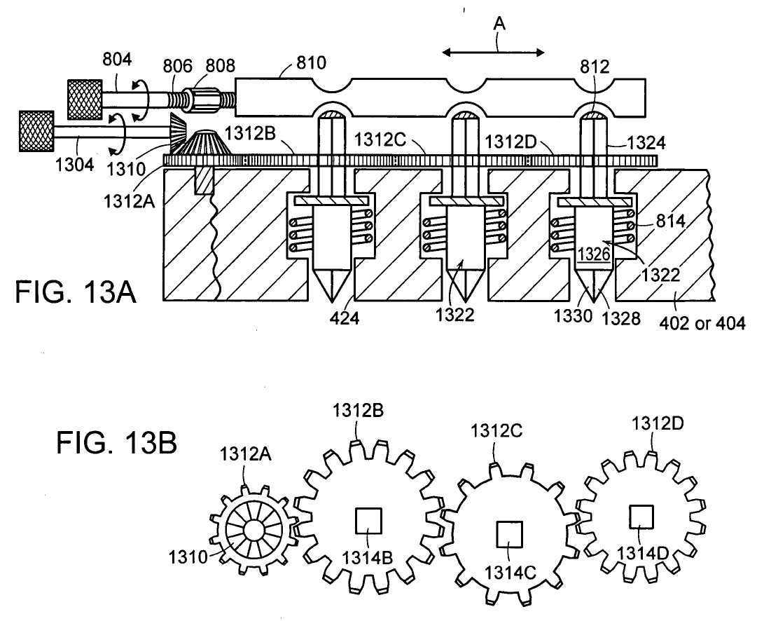

FIGS. 13A and B show an alternative embodiment of the variable thickness cam mechanism for extending the retractable members 800, as shown in FIG. 8. The embodiment depicted in FIGS. 13A and B also allows the retractable members to rotate. This embodiment incorporates the elements of the mechanism 800 as well as the additional elements described below.

Retractable member 1322 includes ridged shaft 1324 connecting upper portion 812 to central body 1326. Retractable member 1322 further includes tip portion 1328 which is provided with ridges 1330 to facilitate drilling through the abutting vertebral bone.

Rotation of actuator 1304, which, in one embodiment, can be a detachable handle, is transmitted to conical crown gear 1310, which, in turn, rotates gears 1312A through 1312D. While FIG. 13A shows four gears 1312, one skilled in the art will appreciate that the actual number of such gears depends on the number of retractable members employed. In the embodiment shown in FIG. 13A, there are three gears, namely 1312B through D that engage ridged shaft 1324 of retractable member 1322, thereby rotating retractable member 1322.

Gears 1312A through D are shown in plan view in FIG. 13B. While gear 1312A engages conical gear 1310, of which it can be a part, gears 1312B through D are provided with polygonal apertures 1314B through D, which engage ridged shafts 1324.

Equivalents

While this invention has been particularly shown and described with references to preferred embodiments thereof, it will be understood by those skilled in the art that various changes in form and details may be made therein without departing from the scope of the invention encompassed by the appended claims.

What is claimed is:

1. A trial intervertebral disk implant, comprising:
a) a first plate;
b) a second plate, adjacent to the first plate;
c) a conformable layer between the first and the second plates; and
d) a sensor within the conformable layer.

2. The trial implant of claim 1 wherein the sensor is a pressure sensor, an angle sensor, a distance sensor, a tissue sensor or a combination thereof.

3. The trial implant of claim 2 wherein the pressure sensor measures a distribution of compression force exerted by the first and the second plates on the conformable layer.

4. The trial implant of claim 2 further including indicating means, disposed within at least one of the first and the second plates or the conformable layer, for indicating a position of the first and the second plates relative to each other.

5. The trial implant of claim 2 further including locating means, disposed within at least one of the first and the second plates or the conformable layer, for locating a position of the trial implant relative to the vertebrae between which said trial implant has been placed.

6. The trial implant of claim 5 wherein the locating means include an ultrasonic transducer.

7. The trial implant of claim 5 wherein the locating means include an impedance sensor.

8. The trial implant of claim 5 wherein the locating means include a infrared proximity sensor.

9. The trial implant of claim 5 further including at least one retractable member, connected to at least one of the first and the second plates, wherein the retractable member can be extended or retracted through an aperture defined by a surface of the plate that is proximal to an abutting vertebra.

10. The trial implant of claim 2 wherein the angle between the first and the second plates is controllably adjustable.

11. A trial intervertebral disk implant, comprising:
a) a first plate;
b) a second plate, adjacent to the first plate; and
c) a sensor at a surface of at least one of the first and the second plates proximal to an abutting vertebra.

12. The trial implant of claim 11 wherein the sensor is a pressure sensor, an angle sensor, a distance sensor or a combination thereof.

13. The trial implant of claim 12 wherein the pressure sensor measures a distribution of a compression force exerted on the plate by an abutting vertebra.

14. The trial implant of claim 11 wherein the pressure sensor is a deformable layer.

15. The trial implant of claim 11 wherein the pressure sensor is a conformable layer.

16. A trial intervertebral disk implant, comprising:
a) a first plate;
b) a second plate, adjacent to the first plate; and
c) indicating means, disposed within at least one of the first or the second plate, for indicating the position of the first and the second plates relative to each other.

17. A trial intervertebral disk implant, comprising:
a) a first plate;
b) a second plate, adjacent to the first plate; and
c) locating means, disposed within at least one of the first or the second plate, for identifying the position of the trial implant relative to the vertebrae between which said trial implant has been placed.

18. A trial intervertebral disk implant, comprising:
a) a first plate;
b) a second plate, adjacent to the first plate;
c) at least one retractable member, connected to at least one of the first and the second plates, wherein the retractable member can be extended or retracted through an aperture defined by a surface of the plate that is proximal to an abutting vertebra; and
d) operating means, in at least one of the first or in the second plate, for extending and retracting the retractable member.

19. The trial implant of claim 18 wherein the trial implant further includes means for rotating the retractable member around a major axis of the retractable member.

20. A method of selecting an artificial intervertebral disk to be inserted between two adjacent vertebral endplates, comprising the steps of:
a) inserting between two adjacent vertebral endplates a trial intervertebral disk implant that includes
(i) a first plate;
(ii) a second plate, adjacent to the first plate;
(iii) a conformable layer between the first and the second plates; and
(iv) a sensor, within the conformable layer, selected from a pressure sensor, an angle sensor, a distance sensor or a combination thereof;
b) measuring a distribution of compression force exerted by the endplates between which the trial implant has been inserted; and
c) comparing the measured distribution of compression force to a distribution that minimizes variation of distribution of compression force while supporting abutting vertebrae in a substantially correct position relative to each other to thereby select an artificial disk.

21. The method of claim 20 further including the steps of:
a) identifying the position of the trial implant relative to the vertebrae between which said trial implant has been placed; and
b) identifying the position of the first and the second plates relative to each other.

22. The method of claim 21 further including identifying the type of tissue proximal to the trial implant.

23. The method of claim 22 wherein the type of tissue is selected form trabecular bone, cortical bone, nerve, collagen and cartilage.

24. The method of claim 21 further including the step of determining tissue density.

25. The method of claim 20 further including a step of adjusting the angle between the upper and the lower end plates to determine the combination of the minimal variation of compression force and a substantially correct relative position of abutting vertebrae.

26. A method of identifying a location between two adjacent vertebral endplates for placement of an artificial intervertebral disk, comprising the steps of:
a) inserting between two adjacent vertebral endplates a trial intervertebral disk implant that includes
(i) a first plate;
(ii) a second plate, adjacent to the first plate;
(iii) at least one retractable member, connected to at least one of the first and the second plate, wherein the retractable member can be extended or retracted through an aperture in a surface of at least one of the first and the second plate proximal to an abutting vertebra; and
(iv) operating means, in at least one of the first and the second plate, for extending and retracting the retractable member; and
b) extending the retractable member from the second plate, thereby indenting at least one of the vertebral endplates between which the trial implant has been placed to identify the location for placement of an artificial intervertebral disk.

27. The method of claim 26 wherein the operating means is at least one of a rack and pinion mechanism, a worm gear drive, a cam mechanism and a hydraulic mechanism.

28. The method of claim 26 wherein the trial implant further includes means for rotating the retractable member around a major axis of the retractable member.

29. The method of claim 28 further including rotating the retractable member around the major axis, thereby drilling a bore in at least one of the vertebral endplates between which the trial implant has been placed.
